(19) United States
(12) Patent Application Publication
(10) Pub. No.: US 2026/0337141 A1
(43) Pub. Date: Oct. 29, 2026

(54) PROCESS FOR RECOVERING VALUE FROM ACRYLIC HEAVIES VIA ESTERS

(71) Applicant: Rohm and Haas Company, Collegeville, PA (US)

(72) Inventors: Joseph F. Dewilde, King of Prussia, PA (US); Mark A. Silvano, Stroudsburg, PA (US); Muhunthan Sathiosatham, Chalfont, PA (US); Lan T.P. Hoang Nguyen, Doylestown, PA (US); James E. Elder, Friendswood, TX (US); Nelson I. Quiros, Houston, TX (US)

(21) Appl. No.: 19/391,395

(22) Filed: Nov. 17, 2025

Related U.S. Application Data
(60) Provisional application No. 63/728,501, filed on Dec. 5, 2024.

(51) Int. Cl. C07C 51/44; C07C 51/48; C07C 51/50; C07C 67/08

(52) U.S. Cl. CPC C07C 51/44; C07C 51/48; C07C 51/50; C07C 67/08

(57) ABSTRACT

A process for recovering acrylic acid from Michael addition products comprises feeding a crude acrylic acid stream to a dehydration tower to produce an overhead stream comprising noncondensables and lights and a bottoms stream comprising acrylic acid and Michael addition products of acrylic acid. At least a portion of the bottoms stream from the dehydration tower is fed to a finishing tower to distill the bottoms stream of the dehydration tower to produce an acrylic acid-rich stream comprising acrylic acid and organic carbonyl compounds, and a bottoms stream comprising acrylic acid and Michael addition products of acrylic acid. At least a portion of the acrylic acid-rich stream is fed to a purification unit to remove the organic carbonyl compounds and form a glacial acrylic acid stream and a bottoms stream comprising heavies. Optionally, an acrylic acid ester is in an esterification process from at least a portion of the acrylic acid in the acrylic acid-rich stream, wherein a waste stream from the esterification process comprises Michael addition products of acrylic acid. At least a portion of the bottoms stream from the finishing tower, the bottoms stream from the purification unit, and/or the waste stream from the esterification process are reacted with a C1 to C8 alkyl alcohol in the presence of an esterification or transesterification catalyst at a temperature ranging from 60 to 120 °C to form an intermediate mixture comprising a C1 to C8 alkyl acrylate and a C1 to C8 alkyl-acryloxy propionate. Excess C1 to C8 alkyl alcohol is removed from the intermediate mixture to form an alkylated heavies stream. The alkylated heavies stream is thermally cracked to produce a mixture of acrylic acid and its corresponding C1 to C8 alkyl ester.

### FIELD OF THE INVENTION

The invention relates to a method for recovering acrylic value from acrylic oligomers.

### BACKGROUND OF THE INVENTION

Acrylic acid and its ester derivatives are used in the manufacture of a broad spectrum of polymer products found across multiple market segments. For example, poly-acrylic acid (PAA) formulations are commonly found in detergent applications, sodium salts of PAA are used as super absorbent polymers (SAP) found in baby diapers and other adult hygiene products. Copolymers of acrylic monomers with methacrylic monomers, styrene, butadiene and other functionalized vinyl monomers are used to manufacture dispersions and resins that are ubiquitous in consumer and industrial products such as architectural, industrial, paper and automotive coatings, performance additives for concrete admixtures and asphalts, textiles, adhesives, sealants and caulks to name just a few applications.

Commercially, acrylic monomer production is dominated by acrylic acid which serves as a platform chemical for a broad range of acrylate derivatives. Acrylic acid may be produced using a variety of different methods. For example, acrylic acid can be made via propylene oxidation, bio-based processes (e.g., using any one of glycerol, 3-hydroxypropionic acid, lactate, lactic acid, etc., as a starting material), the ethylene cyanohydrin process, the acetylene process (i.e., the Reppe process), the β-propiolactone process, acrylonitrile hydrolysis, or coupling carbon dioxide with ethylene or ethane. Industrially, most of the global capacity of acrylic acid is manufactured via the two-stage heterogeneously catalyzed oxidation of propylene over mixed metal oxide catalysts.

The crude acrylic acid (CAA) that is produced by any of the known methods contains Michael addition products, which include dimers, trimers, and higher -mers of acrylic acid. These Michael addition products may reversibly react to form acrylic acid or higher or lower oligomers.

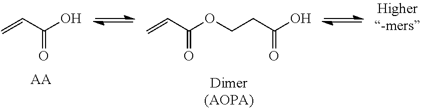

Michael addition products of acrylic acids are also present in waste streams and bottoms streams from acrylic acid purification processes, as well as waste and bottoms streams from ester production processes that use acrylic acid in the formation of the esters, such as, for example, butyl acrylate production.

Dimer crackers are typically used to recover acrylic acid value from the Michael addition products present in these streams.

Attempts have been made to improve acrylic acid recovery in dimer crackers. Some methods utilize strong acids as a catalyst to facilitate recovery of acrylic value.

European Patent EP 1275633 discloses a process for decomposing Michael adducts of acrylic acid by reacting in the presence of an N-oxyl compound to form acrylic acid, acrylic ester, and/or alcohol.

U.S. Pat. No. 7,612,231 discloses a process for recovering acrylic acid from a mixture comprising acrylic acid dimer by carrying out decomposition of the acrylic acid dimer under reduced pressure while recovering acrylic acid by distillation in a process having a retention time at the bottom of the acrylic acid recovering device of 10 to 30 hours.

U.S. Pat. No. 8,308,913 discloses an apparatus for recovering acrylic acid comprising an acrylic acid recovering device having an acrylic acid recovery device having a thin film evaporator integrated with an acrylic acid dimer pyrolysis tank.

U.S. Pat. No. 3,868,410 discloses an esterification process wherein oligomeric byproducts are converted to acrylic acid, an alkanol, or a alkylate of the alkanol by heating the oligomeric byproducts at a temperature of at least 180 °C.

The methods disclosed in the references cited above recover at most only 50 wt % of eligible acrylic acid molar equivalents.

There is a need in the art for an improved process for producing acrylic acid and recovering acrylic acid value from Michael addition products.

### SUMMARY OF THE INVENTION

The invention is directed to a process for recovering acrylic values from Michael addition products comprising:
a) feeding a stream comprising acrylic acid to a dehydration tower to produce an overhead stream comprising noncondensables and lights and a bottoms stream comprising acrylic acid and Michael addition products of acrylic acid;
b) feeding at least a portion of the bottoms stream from the dehydration tower to a finishing tower to distill the bottoms stream of the dehydration tower to produce an acrylic acid-rich stream comprising acrylic acid and organic carbonyl compounds, and a bottoms stream comprising acrylic acid and Michael addition products of acrylic acid;
c) feeding at least a portion of the acrylic acid-rich stream to a purification unit to remove the organic carbonyl compounds and form a glacial acrylic acid stream and a bottoms stream comprising heavies;
d) optionally producing an acrylic acid ester in an esterification process from acrylic acid in the acrylic acid-rich stream, wherein a waste stream from the esterification process comprises Michael addition products of acrylic acid;
e) reacting at least a portion of the bottoms stream from the finishing tower, the bottoms stream from the purification unit, and/or the waste stream from the esterification process with a C1 to C8 alkyl alcohol in the presence of an esterification or transesterification catalyst at a temperature ranging from 60 to 120 °C to form an intermediate mixture comprising a C1 to C8 alkyl acrylate and a C1 to C8 alkyl-acryloxy propionate;
f) removing excess C1 to C8 alkyl alcohol from the intermediate mixture to form an alkylated heavies stream; and
g) thermally cracking the alkylated heavies stream to produce a mixture of acrylic acid and its corresponding C1 to C8 alkyl ester.

### BRIEF DESCRIPTION OF THE DRAWINGS

FIG. 1 is a flow diagram showing the process according to one embodiment of the present invention.

FIG. 2 is a diagram of the chemical reactions associated with the process according to an embodiment of the present invention.

### DETAILED DESCRIPTION OF THE INVENTION

All percentage compositions are weight percentages (wt %), and all temperatures are in °C, unless otherwise indicated. Averages are arithmetic averages unless otherwise indicated. An “average concentration” is the arithmetic average of the concentration entering a region and the concentration exiting the region, where the region is an individual reactor, a reactor system, or a zone within a reactor or reactor system.

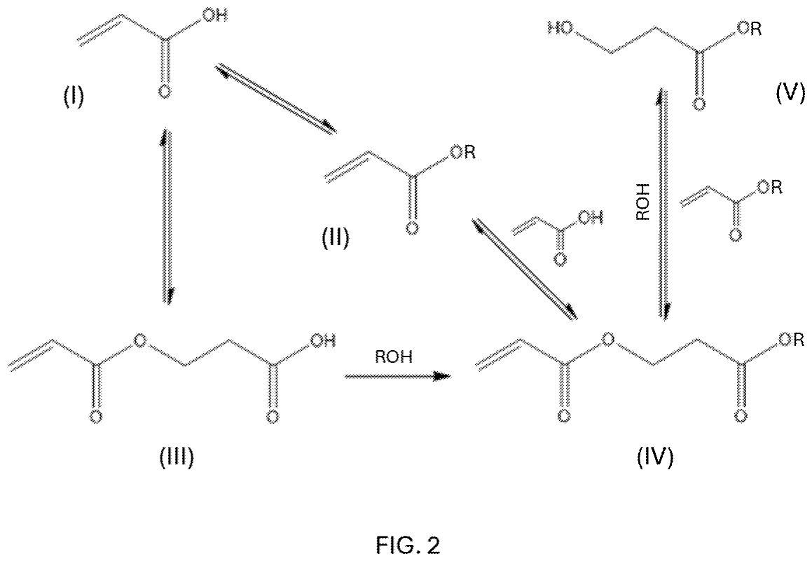

As used herein, the terms “Michael addition products of acrylic acid” and “dimer, trimer, and higher-mers of acrylic acid” refer to oligomers of acrylic acid. The oligomers of acrylic acid can reversibly react to form higher or lower oligomers, as well as acrylic acid. For example, 3-acryloxypropionic acid (AOPA; compound (III) in FIG. 2) may reversibly react to form acrylic acid (compound (I)), as well as the trimer.

The present invention relates to a process for producing acrylic acid and recovering acrylic value from heavy waste byproducts, such as Michael addition products of acrylic acid. As used herein “acrylic value” refers to acrylic acid and esters of acrylic acid that are useful in the production of polymers.

In the present invention, acrylic acid is produced by any known acrylic acid production process. For example, acrylic acid can be made via propylene oxidation, a bio-based process (e.g., using any one of glycerol, 3-hydroxypropionic acid, lactate, lactic acid, etc., as a starting material), an ethylene cyanohydrin process, an acetylene process (i.e., the Reppe process), a β-propiolactone process, an acrylonitrile hydrolysis process, or a process performed by coupling carbon dioxide with ethylene or ethane. The reaction mixture produced during the acrylic acid production process may be processed in one or more unit operations. These operations may include, for example, cooling processes, quenching processes, separations, purifications, etc.

Preferably, the acrylic acid is produced by an exothermic, vapor stage, two-step propylene oxidation process. In a first step, propylene is oxidized in the presence of air and a first mixed metal oxide catalyst to produce acrolein. In a second step, the acrolein is oxidized in the presence of air and a second mixed metal oxide catalyst to produce a gaseous reaction mixture comprising acrylic acid.

The gaseous reaction mixture is cooled or quenched and partially condensed to form a liquid crude acrylic acid stream. The gaseous reaction mixture typically is superheated as it comes from the two-step oxidation process, wherein it contains more heat (energy) than the amount of heat required to vaporize the condensable components of the mixture. Preferably, the cooling step removes essentially all of the amount of superheat from the condensable portion of the gaseous reaction mixture. The cooling step can be conducted directly or indirectly in one or more pieces of equipment. For example, the cooling of the gaseous reaction mixture stream can be initiated in a quench or flash vessel, or can be integrated into the bottom of the dehydration column, with or without column internals. Preferably, the cooling step is initiated or primarily conducted in the dehydration column. The cooling step advantageously is initiated by bringing the gaseous reaction mixture stream into direct contact with a liquid, which preferably has a lower temperature. In one embodiment of the invention, the gaseous reaction mixture is introduced directly into the dehydration column where it is contacted with a liquid having a temperature lower than the temperature of the gaseous reaction mixture in order to at least partially cool the gaseous reaction mixture. In a preferred embodiment of the invention, the gaseous reaction mixture is introduced into the dehydration column and contacted with a spray of a cooler liquid.

Advantageously, the gaseous reaction mixture stream is cooled to a temperature of from about 50 to about 300 °C, preferably from about 70 to about 200 °C, below the boiling point of the majority of the highest-boiling components of the mixture. Preferably, the reaction gas mixture is cooled to a temperature of from about 50 to about 200 °C, more preferably from about 60 to about 180 °C.

The at least partially cooled gaseous reaction mixture stream is dehydrated. The dehydration preferably is conducted in the dehydration column, also referred to as the dehydration tower. The dehydration tower functions to remove the majority of water from the crude acrylic acid stream exiting as the dehydration tower bottoms stream. Advantageously, the dehydration tower is operated such that there is at least a bottoms stream and an overhead stream, and in some cases a side stream. Preferably, at least a portion of the overhead stream is condensed and is returned as a reflux liquid to the dehydration tower. Preferably a portion of the bottoms stream is cooled and used to cool the gaseous reaction mixture.

Preferably, essentially all noncondensables and lights exit the dehydration tower in the overhead stream. Examples of noncondensables present during the production of acrylic acid include, for example, nitrogen, oxygen, CO, carbon dioxide, and unreacted hydrocarbons such as propane and propylene. Advantageously, the overhead stream is introduced into a condenser, and at least a portion of the lights and acrylic acid are condensed and returned to the dehydration tower as a reflux stream.

The dehydration tower functions, at least partially, as a distillation column. However, as noted above, the dehydration column can also serve as a contacting zone for cooling of the gaseous reaction mixture. Preferably, the temperature of the bottoms stream from the dehydration tower is less than about 120 °C. The temperature of the overhead stream from the dehydration tower is at least about 40 °C.

The vent stream from the overhead condenser on the dehydration tower may be partially recycled to the reactor system. The remaining portion of the vent stream is removed from the separation system as a purge stream.

The bottoms stream, or liquid crude acrylic acid stream, from the dehydration tower advantageously is sent to a second column, except that a portion of this stream can be employed to cool the gaseous reaction mixture. In one embodiment of the invention, a portion of the bottoms stream from the dehydration tower is sent to a heat exchanger, which can be a reboiler. However, it is noted that the process can also be operated under conditions such that the heat exchanger is a cooler, depending on whether the process design requires heating or cooling. Preferably, a portion of the bottoms stream from the dehydration tower is fed to a second column. Advantageously, the feed point is near the top of the second column. The second column preferably is a distillation column and is used in conjunction with a reboiler and a condenser.

Preferably, the overhead stream from the second column is sent to a condenser. Preferably, the condenser is operated as a “total condenser” in that essentially all of the overhead stream is condensed. However, it is possible to remove a purge stream of noncondensable compounds from this condenser. A portion of the condensate of the second column is used to provide reflux and the remaining portion of the condensate is returned to the dehydration tower for lights removal. Advantageously, the condensate from the second column condenser is used to cool the gaseous reaction mixture, either as is or after additional heat exchange.

The bottoms stream from the second column advantageously is at least partially sent to the second column reboiler. The remainder of the bottoms stream can be incinerated or can be further treated to recover acrylic values. Preferably, the majority of acrylic acid is recovered from an acrylic acid-rich stream, such as, for example, a side draw stream, from the second column. A portion of the acrylic acid will be present in the bottoms stream of the second column. For example, the acrylic acid-rich stream may comprise technical grade acrylic acid (TGAA) and the bottoms stream may comprise primarily heavies, including Michael addition products of acrylic acid, soluble polymer and polymerization inhibitor residues. Alternatively, the acrylic acid-rich stream may comprise TGAA and the bottoms stream may comprise a lower grade stream of acrylic acid, such as ester grade acrylic acid (EGAA). Preferably, the acrylic acid-rich stream comprises at least 95 wt %, more preferably at least 96 wt %, even more preferably at least 97 wt %, and still more preferably at least 98 wt % acrylic acid based on the total weight of the acrylic acid-rich stream.

The process of the invention can remove the acrylic acid-rich stream at a point above or below the point where the liquid crude acrylic acid stream is fed to the second column, depending on the purity desired, i.e. the acrylic acid-rich stream can be removed above or below the feed in the second column. The acrylic acid-rich stream may be removed as a liquid or vapor.

The temperature and pressure in the second column can be determined according to design considerations well-known to those skilled in the art. Preferably, the second column is operated pressures so that the bottoms temperature is less than 120 °C. More preferably, the second column is operated at pressures so that the bottoms temperature is less than 100 °C. The advantage of allowing the second column to operate at lower temperatures is to minimize undesired dimer, oligomer and/or polymer formation. Advantageously, the temperature of the overhead stream as it leaves the second column is from about 40 to about 90 °C when producing acrylic acid and operating the second column at a head pressure of from about 40 to about 500 mm Hg. The temperature of the bottoms stream from the second column advantageously is from about 60 to about 100 °C when producing acrylic acid.

The design details of the dehydration column and of the second column, including their operating conditions such as temperatures, pressures, flow rates, equipment sizing including column height and diameters, choice of materials of construction, arrangement and choice of type of auxiliary equipment such as heat exchangers and pumps, choice and arrangement of column internals, and location of piping including take-off streams, can readily be determined by those skilled in the art according to well-known design considerations. Examples of distillation column configurations that can be used in the process of the invention include, for example, packed columns, trayed columns, divided wall columns, multi-stage devolatilizers, and the like. Any type of tray can be employed, including bubble trays, valve trays, cross flow trays, dual flow trays, and combinations thereof for those skilled in the art. Similarly, if packing is employed, any type of packing can be used, including randomly- or regularly-spaced packing. In a preferred embodiment of the invention, the dehydration column comprises packing in its upper section, and in its lower section provides for direct cooling of the incoming gaseous reaction mixture. Surge tanks optionally can be employed within the separation system such as, for example, at one or more locations between the dehydration column and the second column.

Steel alloys such as 316 SS and other higher alloys may be used as the material(s) of construction for the process equipment by using criteria well known to those skilled in the art for process equipment to be used in the distillation of corrosive streams. Alternatively, the process equipment can be partially constructed using copper or copper-containing alloys, such as various alloys sold under the name Monel. While not wishing to be bound by any theory, it is the believed that the presence of copper inhibits the undesired polymerization of acrylic acid.

The use of inhibitors is preferred in the process of the invention, regardless of the choice of materials of construction. Various compounds are well known to inhibit the reaction of acrylic acid, and are commercially available. Examples of preferred inhibitors include complexes and salts of manganese compounds, copper complexes and salts thereof, hydroquinone (HQ), monomethyl ether hydroquinone (MEHQ), phenothiazine (PTZ), 2,2,6,6-tetramethyl-1-piperidinyloxy (TEMPO), its derivatives, and related compounds such as 4-hydroxy TEMPO. Combinations of inhibitors can be employed that offer synergistic interactions and provide more effective polymerization inhibition than the additive performance of the individual inhibitors. In a preferred embodiment of the invention, a mixture of a source of soluble manganese ions, such as manganese acetate, and 4-hydroxy TEMPO are employed in the dehydration column as an inhibitor. Hydroquinone can also be added to this inhibitor mixture. It is also preferred, as is well-known in the art, to employ a molecular oxygen or air to the second column, as oxygen is known to be a polymerization retarder. The inhibitor is employed in an amount sufficient to prevent or reduce the polymerization of acrylic acid, as is well known to those skilled in the art.

Preferably, the dehydration tower and second column form a coupled distillation tower system. The coupled distillation tower system comprises the dehydration tower and a finishing tower (i.e., the second column). Other suitable process configurations are possible and are known to those skilled in the art which use intermediate tanks to contain certain process streams prior to being fed to each tower.

Preferably, at least a portion of the acrylic acid in the acrylic acid-rich stream is fed to an esterification process to convert acrylic acid to an ester of acrylic acid. Preferably, the acrylic acid ester formed in the esterification process is selected from the group consisting of methyl acrylate, ethyl acrylate, butyl acrylate, and 2-ethyl hexyl acrylate. More preferably, the acrylic acid ester formed in the esterification process is butyl acrylate. The esterification process may comprise one or more unit operations to purify the acrylic acid ester to produce a product stream and a waste stream comprising heavies including Michael addition products of acrylic acid.

The acrylic acid in the acrylic acid-rich stream of the second column comprises residual organic carbonyl compounds that are byproducts of the heterogeneously-catalyzed oxidation of propylene, such as maleic acid, maleic anhydride, propionic acid, acetic acid, formaldehyde, furfuraldehyde, benzaldehyde, acrolein, protoanemonin, acrylic acid dimer among others. High levels of maleic anhydride, aldehydes and other similar species, can render the acrylic acid unsuitable for use in certain products, such as the formation of super absorbent polymers (SAP), which require low aldehyde concentrations. Examples of the residual organic carbonyl compounds include furfural, benzaldehyde, acrolein, maleic acid, and formaldehyde. The amount of these compounds varies according to reactor catalyst performance and may be present individually in the acrylic acid-rich stream in amounts ranging from 10 ppm to 2000 ppm depending on the individual component.

To further purify the acrylic acid of the acrylic acid-rich stream of the second column and reduce the amount of residual organic carbonyl compounds, and preferably to reduce the amount of residual aldehydes, at least a portion of the acrylic acid-rich stream is fed to a purification unit to remove at least a portion residual organic carbonyl compounds and to produce a glacial acrylic acid (GAA) stream. Preferably, the acrylic acid-rich stream of the second column is fed to a surge tank before proceeding to the purification unit. The surge tank may help manage fluctuations in flow rates or pressure, and may also be used as to divert the acrylic acid-rich stream into one or more streams. For example, a first portion of the acrylic acid-rich stream may be fed to purification unit, and a second portion of the acrylic acid-rich stream may be fed to a downstream unit, such as an ester unit for manufacturing acrylic acid esters from the side draw stream. Preferably, at least a portion of the acrylic acid-rich stream is fed to the purification unit and a remainder of the side draw stream is fed to an ester unit for manufacturing acrylic acid esters.

In one embodiment, the purification unit comprises a reactive distillation tower to form a glacial acrylic acid (GAA) stream. A chemical additive is fed to the reactive distillation tower to react with the residual organic carbonyl compounds and form stable carbonyl adducts that are separable from the acrylic acid by distillation. The chemical additive may be added to the tower itself, or to the feed stream to the tower. The additive can be added directly to said feed or via a device that will efficiently mix the additive with the acrylic acid stream so as to maximize the formation of the adducts of the additive with the unwanted carbonyl compounds with the lowest practical amount of chemical additive compound added. These heavy carbonyl adducts are then partitioned into the bottoms of the reactive distillation column. GAA is removed from the reactive distillation column as a distillate. As some chemical additives may generate water as a byproduct of their reaction with said residual carbonyl compounds and water being a light component relative to acrylic acid, GAA with a lower water content may alternately be drawn from the reactive distillation tower as a side draw stream. The GAA side draw stream may be removed and subjected to cooling through a heat exchanger prior to collection and storage.

The chemical additive may comprise compounds such as amines including ethanolamine, ethylenediamine, hydrazine, hydrazine hydrate and its derivatives, aminoguanidine and its derivatives, mono-phenyl-amines such as aniline and its analogs which may have a plurality of substituents on the ortho, meta and para positions of the phenyl ring such as methyl, methoxy, ethyl, ethoxy and other substituents, phenylenediamine isomers such as ortho, para and meta-phenylenediamines and their derivatives, and other similar compounds that will react with the carbonyl group of the compound converting it to a stable, heavy compound separable from acrylic acid by distillation. The additive compound may be used individually or in mixtures or combination thereof with other additives.

In an alternative embodiment, the purification unit comprises a crystallizer to form a glacial acrylic acid (GAA) stream. The crystallizer may use any known crystallization methods, such as, for example, static, falling film, suspension, or others, as well as combinations thereof. Preferably, the crystallizer comprises a suspension crystallizer. The crystallizer removes residual organic carbonyl compounds from the acrylic acid-rich stream. In addition to the acrylic acid product, the crystallizer also forms a mother liquor. The mother liquor may comprise acrylic acid, dimers, trimers, and higher oligomers, as well as a relatively high level of polymerization inhibitors. Depending on the crystallization technology, the mother liquor may be subjected to additional crystallization cycles to increase the yield of GAA and/or reduce the amount of residual carbonyl or aldehydes, or it may be recycled back to the finishing tower as supplementary recycle inhibitor or may be disposed of by incineration.

The GAA produced in the purification unit preferably comprises less than 20 ppm of furfural, benzaldehyde, formaldehyde, and acrolein combined, i.e., the total content of furfural, benzaldehyde, formaldehyde, and acrolein in the GAA is less than 20 ppm. More preferably, the GAA comprises less than 10 ppm of furfural, benzaldehyde, formaldehyde, and acrolein combined, and still more preferably, less than 5 ppm of furfural, benzaldehyde, formaldehyde, and acrolein combined.

In at least one preferred embodiment, the GAA produced in the purification unit comprises less than 5 ppm of each of furfural, benzaldehyde, formaldehyde, and acrolein. More preferably, the GAA comprises less than 3 ppm of each of furfural, benzaldehyde, formaldehyde, and acrolein, and even more preferably less than 2 ppm of each of furfural, benzaldehyde, formaldehyde, and acrolein.

The bottoms stream of the second column, the bottoms of the reactive distillation column or the mother liquor from the crystallizer, and/or the waste stream from the esterification process are combined to form a heavies stream. The heavies stream comprises acrylic acid, the dimer of acrylic acid (3-(acryloyloxy) propanoic acid or AOPA), acrylic acid trimer, and acrylic acid tetramer. Higher oligomers of acrylic acid may also be present. The dimer is preferably the majority component of the Michael addition products. More preferably, the dimer is the majority component in the heavies stream.

Acrylic value recovery is performed by reacting, in the liquid phase, the heavies stream with a C1 to C8 alkyl alcohol (i.e., ROH, where R is a C1 to C8 alkyl group) to form an intermediate mixture comprising a C1 to C8 alkyl acrylate and a C1 to C8 alkyl-acryloxy propionate.

When reacted with a C to C8 alkyl alcohol (shown as ROH in FIG. 2, where R is a C1 to C8 alkyl group), corresponding C1 to C8 alkyl esters of the acrylic acid (i.e., C1 to C8 alkyl acrylates, compound (II)) and its Michael addition products (compound (IV)) are formed, as shown in FIG. 2.

The alkoxy-3-acryloyloxypropanoic acid of formula (compound (IV)) may further react with the C1 to C8 alkyl alcohol to form an alkyl-3-hydroxy propionate (compound (V)) as well as oligomers thereof. To avoid formation of the alkyl-3-hydroxy propionate (compound (V)) and its oligomers, the reaction of the stream comprising acrylic acid and its Michael addition products with a C1 to C8 alkyl alcohol is conducted at a temperature between 6° and 120 °C, preferably from 65 to 100 °C, and more preferably from 70 to 85 °C.

The reaction of the acrylic acid and its Michael addition products with a C1 to C8 alkyl alcohol is catalyzed with an esterification or transesterification catalyst. Preferably, the esterification or transesterification catalyst is a homogeneous acid catalyst. Examples of suitable acid catalysts include hydrochloric acid, sulfuric acid, p-toluenesulfonic acid, methane sulfonic acid, and phosphoric acid. Preferably, the catalyst is sulfuric acid.

The C1 to C8 alkyl alcohol is preferably selected from methanol, ethanol, butanol, and 2-ethyl hexyl alcohol. More preferably, the C1 to C8 alkyl alcohol is butanol.

Optionally, water may be removed from the intermediate mixture before further processing. The water may be removed using phase separation. The aqueous phase may contain impurities, as well as some of the acid catalyst.

Excess C1 to C8 alkyl alcohol from the intermediate mixture to form an alkylated heavies stream. Excess C1 to C8 alkyl alcohol may be removed by evaporation. In one embodiment, the step of removing excess C1 to C8 alkyl alcohol can be performed in the same vessel as the reaction of the C1 to C8 alkyl alcohol with the stream comprising acrylic acid and Michael addition products of acrylic acid. In an alternative embodiment, the removal of excess C1 to C8 alkyl alcohol takes place in a different vessel. For example, the excess C1 to C8 alkyl alcohol may be removed using a flash pot, a distillation column, or other evaporative separation unit.

Any of the C1 to C8 alkyl acrylates formed by the reaction of acrylic acid with the C1 to C8 alkyl alcohol may also be removed with the excess C1 to C8 alkyl alcohol.

Preferably, removal of the excess C1 to C8 alkyl alcohol is performed at a temperature less than 100 °C, more preferably less than 90 °C, and even more preferably less than 80 °C.

Preferably, removal of the excess C1 to C8 alkyl alcohol is performed under vacuum (i.e., less than atmospheric pressure). For example, the excess C1 to C8 alkyl alcohol may be removed at a pressure of 50 mmHg.

Removal of the excess C1 to C8 alkyl alcohol results in an alkylated heavies stream.

The removed excess C1 to C8 alkyl alcohol may be condensed and optionally recycled to the first step of the process.

The alkylated heavies stream is thermally cracked to form a mixture of acrylic acid and its corresponding C1 to C8 alkyl ester (i.e., C1 to C8 alkyl acrylate). The alkylated heavies stream is preferably cracked at an elevated temperature of at least 180 °C, more preferably at least 190 °C, and even more preferably at least 200 °C.

In one embodiment, the alkylated heavies stream is thermally cracked by recirculating the alkylated heavies stream between a pressurized liquid phase reactor and an evacuated flash pot, where the pressurized liquid phase reactor is operated at a higher temperature than the evacuated flash pot. For example, the pressurized liquid phase reactor may be operated at a temperature greater than 180 °C and the evacuated flash pot may be operated at a temperature less than 140 °C. It is believed that the combination of the pressurized liquid phase reactor and the evacuated flash pot aids cracking of the heavy esters by removing the acrylic acid and C1 to C8 alkyl acrylates formed in the pressurized liquid phase reactor to drive the reversible reaction in the direction of additional cracking of the heavy esters.

The acrylic acid and C1 to C8 alkyl acrylates produced may be condensed and collected.

Preferably, a polymerization inhibitor is added at any point in the process or already present in the heavies stream by virtue of being added during a previous distillation/separation step.

The present invention surprisingly enables the recovery of a significant amount of the Michael addition products of acrylic acid from the heavies stream. For example, when butanol is used as the C1 to C8 alkyl alcohol, 90 wt % or more of the Michael addition products of acrylic acid in the acrylic heavies may be recovered as acrylic acid or butyl acrylate. This represents a significant improvement over conventional dimer cracking methods.

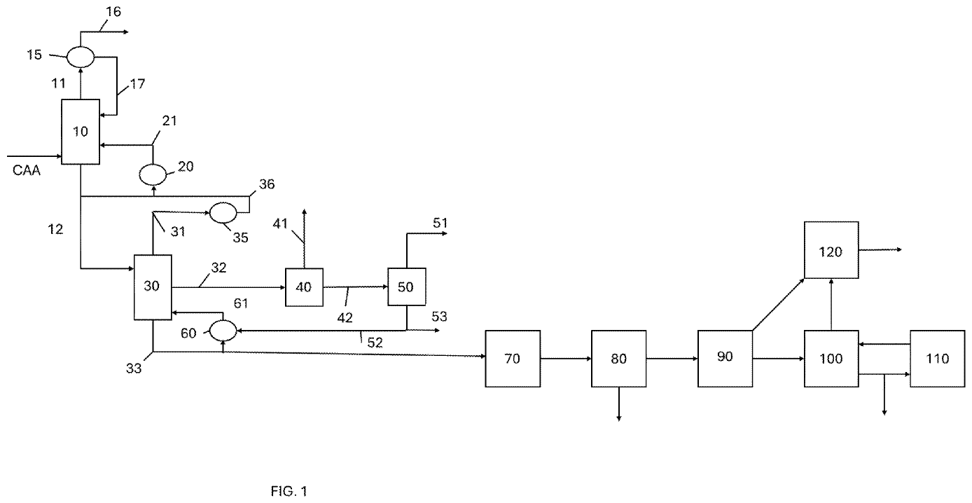

One embodiment of the present invention is shown in FIG. 1. A crude acrylic acid stream CAA is fed to a dehydration tower 10, where the gaseous crude acrylic acid stream is quenched and condensed. The lights 11 from dehydration tower 10 are sent to condenser 15. Condensable materials 17 are returned to dehydration tower 10 as reflux. Lights and noncondensable materials 16 may be vented or incinerated. A portion of the bottoms stream 12 is sent to a heat exchanger 20 and liquid stream 21 exiting the heat exchanger 20 is returned to dehydration tower 10 as a cooling liquid.

The remainder of bottoms stream 12 is fed to second column 30, which functions as a distillation column. An overhead stream 31 from second column 30 is condensed in condenser 35 and the condensed overhead stream 36 is returned to dehydration tower 10. A side draw 32 comprising primarily acrylic acid is removed from second column 30. A bottoms stream 33 comprising acrylic acid and heavies exits the bottom of finishing column 30.

Side draw 32 is fed to surge tank 40, which is then split into two streams 41, 42 exiting surge tank 40. First stream 41 is fed to an ester unit (not shown) where a corresponding acrylic acid ester is produced. Second stream 42 is fed to a crystallizer 50. A product stream 51 exiting crystallizer 50 comprises GAA. Bottoms stream 52 from crystallizer 50 contains acrylic acid, oligomers of acrylic acid and residual polymerization inhibitors. Bottoms stream 52 may be returned to reboiler 60 to be recycled to second column 30 as stream 61. Alternately, the bottoms stream of crystallizer 50 may be sent to a tank (not shown) from which stream 53 can be recycled to the crystallizer or sent to incineration as waste oil.

The bottoms stream 33 comprising acrylic acid and Michael addition products of acrylic acid is fed to a reactor 70, along with a C to C8 alkyl alcohol and an acid catalyst. The intermediate stream exiting reactor 70 is fed to a phase separator 80 to remove the aqueous phase. The remaining intermediate stream is fed to a flash pot 90 to remove excess C1 to C8 alkyl alcohol. Alternatively, the excess C1 to C8 alkyl alcohol may be removed in reactor 70 as a vapor stream. The vapor from the flash pot 90 is sent to a condenser 120. The alkylated heavies are sent to an evacuated flash pot 100, which is in recirculation with pressurized liquid phase reactor 110. Heavies waste can be removed from the recirculating alkylated heavies stream. The vapor phase containing the recovered acrylic acid and C1 to C8 alkyl acrylate is removed from the evacuated flash pot 100. As shown in FIG. 1, a common condenser 120 is used to condense both the recovered C1 to C8 alkyl alcohol, acrylic acid and C1 to C8 alkyl acrylates. However, separate condensers can also be used.

### Example

Step 1—Liquid phase reaction at lower temperature: a 500 ml glass jacketed flask was charged with 380 g of mixed reactor material comprising 50 wt % acrylate heavies feed, 45 wt % butanol, and 5 wt % sulfuric acid. The mixture was stirred with an impeller and heated to 75 °C under atmospheric pressure. Once at temperature, the same ratio of feed material was fed and reactor fluid was drained to maintain a residence time of 150 min. This mixture was fed until 6000 g of total feed was fed (3000 g of acrylate heavies feed).
Step 2—Removal of aqueous layer formed in Step 1 (optional): The collected bottoms were piecewise placed in a standard glass separatory funnel. Using a flashlight to distinguish the phase interface, the bottom aqueous layer was removed and separated. Each layer was then analyzed for organic composition via gas chromatography (GC) and sulfuric content via titration.
Step 3—Light Compound Flash: The organic portion of Step 2 was fed to a glass three-neck flask acting as a distillation pot at 67 °C and 50 mmHg absolute. An additional stream of water in 0.05:1 mass ratio with the organic feed was also fed to the unit. The liquid level was adjusted such that a feed-based residence time of 15 minutes was maintained. The distillate was gravity separated into organic and aqueous phases after condensing in a water-cooled condenser. As with step 2, both distillate phases as well as the reactor bottoms were analyzed via GC and titration to determine composition.
Step 4—Recirculating High Temperature Reaction and Flash Pot: The setup in this step consists of a glass three-neck flask used for the flashing of product material (similar to step 3) and a liquid phase tube packed with glass spheres heated by an external oil heating bath loop. Fluid was set up to rapidly recirculate between the flash vessel and the packed bed to maintain a well-mixed behavior for the two vessels (recirculation time ~15 min). The void volume of this packed bed was such that it was 3-4% of the liquid level in the flash pot. The flash pot was kept at 80 °C and 50 mmHg absolute. The liquid phase reactor was kept at 200 °C and >300 psi to maintain fluid in liquid phase. The outlet of the liquid phase reactor was fed through a water cooled heat exchanger to cool the fluid prior to depressurization over a back pressure regulator near the recirculation entrance. A small bleed was maintained to control fluid level at the bottom of the flash pot-representing the bottoms of the combined process. The bottoms from Step 3 was fed with water (0.1:1 mass ratio water:Step 3 bottoms) to maintain a 160 min feed-based residence time in the combination of the two vessels. As with Step 3, the distillate from the flash vessel portion was condensed and phase separated. Following the other steps, GC and titration measured the compositions of the two distillate layers and the bottoms.

After performing Step 1 and 2 as described above with a total cumulative feed of 3000 g acrylate heavies feed, 2700 g of butanol, and 300 g of sulfuric acid. Approximately 5600 g of organic layer and 130 g of aqueous layer (96% mass balance across these two steps) were collected. Then after taking 5000 g of this organic layer and a cumulative water feed of 270 g of water in Step 3, 1770 g of organic distillate, 630 g of aqueous distillate and 3140 g of process bottoms (mass balance around 105%) were recovered. Finally, 340 g of the bottoms from Step 3 and with 35 g of additional cumulative water feed of 35 g were treated through the process described in Step 4. As a result, 300 g of organic distillate, 57 g of aqueous distillate, and produced only 31 g of bottoms (total mass balance of ~102%) were recovered.

TABLE 1
All Compositions in wt Fraction

| | | | | | | | | | | |
|---|---|---|---|---|---|---|---|---|---|---|
| | | | | | | Acrylic | | | | |
| | | Higher | | Butyl-3- | Butyl-3- | Dimer | | | | |
| Acrylic | Acrylate | Acrylate | Butyl | butoxy | hydroxy | Butyl | n- | Dibutyl | | Sulfuric |
| Acid | Dimer | Heavies | Acrylate | Propionate | Propionate | Ester | Butanol | Ether | Water | Acid |

Steps 1 and 2 Organic Layer

| | | | | | | | | | | |
|---|---|---|---|---|---|---|---|---|---|---|
| 0.057 | 0.020 | 0.000 | 0.286 | 0.004 | 0.061 | 0.317 | 0.180 | 0.000 | 0.038 | 0.034 |

Steps 1 and 2 Aqueous Layer

| | | | | | | | | | | |
|---|---|---|---|---|---|---|---|---|---|---|
| 0.031 | 0.000 | 0.000 | 0.001 | 0.000 | 0.001 | 0.000 | 0.022 | 0.000 | Balance | 0.342 |

Step 3 Organic Distillate

| | | | | | | | | | | |
|---|---|---|---|---|---|---|---|---|---|---|
| 0.047 | 0.000 | 0.000 | 0.718 | 0.000 | 0.001 | 0.000 | 0.257 | 0.000 | 0.027 | 0.000 |

Step 3 Aqueous Distillate

| | | | | | | | | | | |
|---|---|---|---|---|---|---|---|---|---|---|
| 0.012 | 0.000 | 0.000 | 0.001 | 0.000 | 0.000 | 0.000 | 0.033 | 0.000 | Balance | 0.000 |

Step 3 Bottoms

| | | | | | | | | | | |
|---|---|---|---|---|---|---|---|---|---|---|
| 0.034 | 0.019 | 0.000 | 0.197 | 0.010 | 0.093 | 0.449 | 0.091 | 0.000 | 0.013 | 0.101 |

Step 4 Organic Distillate

| | | | | | | | | | | |
|---|---|---|---|---|---|---|---|---|---|---|
| 0.083 | 0.000 | 0.000 | 0.754 | 0.005 | 0.003 | 0.004 | 0.063 | 0.037 | 0.017 | 0.000 |

Step 4 Aqueous Distillate

| | | | | | | | | | | |
|---|---|---|---|---|---|---|---|---|---|---|
| 0.024 | 0.000 | 0.000 | 0.002 | 0.000 | 0.000 | 0.000 | 0.020 | 0.000 | Balance | 0.000 |

Step 4 Bottoms

| | | | | | | | | | | |
|---|---|---|---|---|---|---|---|---|---|---|
| 0.119 | 0.004 | Balance | 0.092 | 0.032 | 0.041 | 0.068 | 0.184 | 0.002 | 0.023 | 0.050 |
| | | Assumed | | | | | | | | |

The compositions (wt fraction) of each of these streams is included in the table above. Using these and the recovered masses, we were able to define what fraction of “C3” portions are present in the remaining bottoms for each steps: defined as the number of moles of C3 fragments in the bottoms (for example 2× the number of moles of acrylic dimer) compared to the sum total number of moles of C3 units fed to the Step described. For the bottoms of step 4, because we did not have complete analytical closure, we made the conservative assumption of assuming all unaccounted composition (40 wt %) was lost C3, making the reported yields the lower end interpretation of the data. Doing so, we calculated the following:
Fractional C3 loss present in the aqueous stream of Steps 1 and 2~0.0015
Fraction of C3 fed to Step 3 present in the bottoms of Step 3~0.61
Fraction of C3 fed to Step 4 in the bottoms of Step 4~0.11
   To represent the total lost C3 we multiplied the last two of these numbers (as the bottoms of Step 3 was fed to Step 4) and add the first (as the aqueous stream was not fed downstream). There was found an overall fractional loss of ~0.067, or in other words, an acrylate value recovery efficiency of >93%.

1. A process for recovering acrylic values from Michael addition products comprising:
a) feeding a stream comprising acrylic acid to a dehydration tower to produce an overhead stream comprising noncondensables and lights and a bottoms stream comprising acrylic acid and Michael addition products of acrylic acid;
b) feeding at least a portion of the bottoms stream from the dehydration tower to a finishing tower to distill the bottoms stream of the dehydration tower to produce an acrylic acid-rich stream comprising acrylic acid and organic carbonyl compounds, and a bottoms stream comprising acrylic acid and Michael addition products of acrylic acid;
c) feeding at least a portion of the acrylic acid-rich stream to a purification unit to remove the organic carbonyl compounds and form a glacial acrylic acid stream and a bottoms stream comprising heavies;
d) optionally producing an acrylic acid ester in an esterification process from at least a portion of the acrylic acid in the acrylic acid-rich stream, wherein a waste stream from the esterification process comprises Michael addition products of acrylic acid;
e) reacting at least a portion of the bottoms stream from the finishing tower, the bottoms stream from the purification unit, and/or the waste stream from the esterification process with a C1 to C8 alkyl alcohol in the presence of an esterification or transesterification catalyst at a temperature ranging from 60 to 120 °C to form an intermediate mixture comprising a C1 to C8 alkyl acrylate and a C1 to C8 alkyl-acryloxy propionate;
f) removing excess C1 to C8 alkyl alcohol from the intermediate mixture to form an alkylated heavies stream; and
g) thermally cracking the alkylated heavies stream to produce a mixture of acrylic acid and its corresponding C1 to C8 alkyl ester.

2. The process of claim 1, wherein step e) comprises reacting at least a portion of the waste stream from the esterification process with the C1 to C8 alkyl alcohol.

3. The process of claim 1- or 2, wherein the C1 to C8 alkyl alcohol is selected form the group consisting of methanol, ethanol, butanol, and 2-ethyl hexyl alcohol.

4. The process of claim 3, wherein the C1 to C8 alkyl alcohol is butanol.

5. The process of claim 1, wherein step a) is performed at a temperature ranging from 65 to 100 °C.

6. The process of claim 1, wherein removing excess C1 to C8 alkyl alcohol from the intermediate mixture is performed at temperature less than 100 °C.

7. The process of claim 6, wherein removing excess C1 to C8 alkyl alcohol from the intermediate mixture is performed at temperature less than 90 °C.

8. The process of claim 1, wherein removing excess C1 to C8 alkyl alcohol from the intermediate mixture is performed in a flash pot, distillation column, or other evaporative separation unit.

9. The process of claim 1, wherein removing excess C1 to C8 alkyl alcohol from the intermediate mixture is performed under vacuum.

10. The process of claim 1, wherein thermally cracking the alkylated heavies stream comprises recirculating the alkylated heavies stream between a pressurized liquid phase reactor and an evacuated flash pot, wherein the pressurized liquid phase reactor is operated at a higher temperature than the evacuated flash pot.

11. The process of claim 10, wherein the pressurized liquid phase reactor is operated at a temperature greater than 180 °C and the evacuated flash pot is operated at a temperature less than 140 °C.

12. The process of claim 11, wherein the pressurized liquid phase reactor is operated at a temperature greater than 190 °C.

13. The process of claim 1, further comprising a phase separation step to remove water from the intermediate mixture prior to removing the excess C1 to C8 alkyl alcohol.

14. The process of claim 1, wherein the purification unit comprises a crystallizer.

15. The process of claim 1, wherein the purification unit comprises a reactive distillation column and a chemical additive is fed to the reactive distillation column to react with the organic carbonyl compounds in the side draw stream to form heavy carbonyl adducts that are reacted with the C1 to C8 alkyl alcohol.

16. The process of claim 1, wherein the acrylic acid in the stream fed to the dehydration tower is produced by a method selected from the group consisting of propylene oxidation-based process, a bio-based process, an ethylene cyanohydrin-based process, an acetylene-based process, a β-propiolactone-based process, an acrylonitrile hydrolysis-based process, or a process performed by coupling carbon dioxide with ethylene or ethane.
